(19) United States
(12) Patent Application Publication
(10) Pub. No.: US 2026/0257066 A1
(43) Pub. Date: Sep. 3, 2026

(54) NERVE REPAIR IMPLANT

(71) Applicants: SORBONNE UNIVERSITE, Paris (FR); CENTRE NATIONAL DE LA RECHERCHE SCIENTIFIQUE, Paris (FR); UNIVERSITE PARIS CITE, Paris (FR)

(72) Inventors: Ahmed Hamraoui, Paris (FR); Claire Legay, Paris (FR); Océane Sénépart, Paris (FR)

(21) Appl. No.: 19/486,825

(22) PCT Filed: May 24, 2023
(86) PCT No.: PCT/IB2023/000262

§ 371 Date: Nov. 21, 2025

(51) Int. Cl. A61N 1/36; A61L 27/34; A61L 27/52

(52) U.S. Cl. CPC A61N 1/36103; A61L 27/34; A61L 27/52; A61L 2430/32

(57) ABSTRACT

The present invention relates to a nerve repair implant for the peripheral nervous systems comprising a tube, electrodes, a muff and a gel.

### FIELD OF INVENTION

The present invention relates to an implantable device that can be used for central and peripheral nerve repair for promoting nerve growth and nerve regeneration.

### BACKGROUND OF INVENTION

Crushes or sections of peripheral nerves are common accidents and are very disabling. An emerging approach to facilitate nerve regeneration within nerve guides is a therapy based on the application of an electric field (EF). Any discussion of applied exogenous EFs must start with a consideration of the biological electrical environment. Ion transport generates potentials of tens of millivolts. Inside tissues, the EF results from a low resistance and the resulting current flow through the extracellular spaces. EFs play a major role during the development and the reparation of a variety of tissues and in particular were shown to drive and direct axonal growth. Thus, for example the disturbance of such endogenous EFs generated in chicken embryo causes abnormalities in the development of both the nervous and skeletal muscle systems. However, the underlying mechanisms of neuron response to an exogenous EF are unknown or poorly understood and remains largely speculative. This is mainly due to the absence of comprehension of the physical basis of the phenomena and a lack of understanding of the associated fundamental concepts. Numerous studies have addressed the role of EF in the nervous system. For instance, the combination of direct (in situ) electric field stimulation, chemical gradients and walking rehabilitation was successfully used in a partially severed spinal cord (Restoring Voluntary Control of Locomotion after Paralyzing Spinal Cord Injury Rubia, van den Brand et al. Science 336, 1182 (2012); DOI: 10.1126/science. 1217416).

A major weakness of previous studies is the use of a global EF generated in conducting polymers or via Ag/AgCl electrodes immersed in the culture medium connected via agar-salt bridges. These experimental setups do not discriminate effects coming from the ions in solution, which are driven by the EF, and effects originating from the substrate (adhesion effect). Another disadvantage of these experiments is the loss of orientation of the neurite outgrowth a few hours after stimulation. This cessation of alignment with EF is obviously due to the dissipation of the ion gradient after deletion of EF. For instance, electrodes are in direct contact with culture media and axons, which produce electrolysis and heating (HONEGGER, Thibault, THIELEN, Moritz I., FEIZI, Soheil, et al. Microfluidic neurite guidance to study structure-function relationships in topologically-complex population-based neural networks. Scientific reports, 2016, vol. 6, no 1, p. 1-10.). Another experimental setup, contactless, uses a magnetic field to induce an ion current in the culture medium (AHIRWAR, Dinesh K., NASSER, Mohd W., JONES, Travis H., et al. Non-contact method for directing electrotaxis. Scientific reports, 2015, vol. 5, no 1, p. 11005). This ions current induces the same effect as agar or salt bridges, i.e., a global movement of ions inside the cell culture medium, which is unrealistic in a physiological context. All these procedures used exogenous electrical stimulation to promote nerve regeneration and have got only limited success.

Surprisingly, applicant realized that the spatial distribution of the energy of adhesion on a rigid substrate is critical for neuritogenesis and evidenced that the differentiation of PC12 cells does not depend critically on the overall amplitude (high or low) of the adhesion energy but is strongly sensitive to the presence different contributions to the adhesion energy such as dispersive (long range) and non-dispersive (short range) interactions, the latter including those of electrical origin. Therefore, the presence of a local EF induces a local modification of the energy of adhesion that, in turn, have an impact on neuritogenesis.

To overcome the defects of the prior art, the present invention provides an implant for the nerve repair or growth which leads to a local change of surface energy modulation—when EF is applied. In addition, the intrinsic stiffness of the implant undergoes slight modifications when EF is applied, thereby influencing cell differentiation and growth.

The goal of the present invention is to provide an implant for nerve repair which provides optimal growth and guidance support to the nerve.

It is also a goal of the present invention to provide an implant for nerve repair which is easy to implant with standardized procedures.

### SUMMARY

This invention thus relates to nerve repair implant comprising
an insulating tube having an internal surface and an external surface, said tube forming a channel, said channel comprising a gel,
at least two electrodes designed on the external surface of the tube,
an insulating muff surrounding the tube and in direct contact with the tube,
wherein the gel is adapted to accommodate an axon and ensure growth of the axon, and

In an embodiment, the gel fills the channel. Alternatively, the gel coats the internal surface of the tube.

In an embodiment, the tube has a length in the range 15 mm-85 mm, preferably 30 mm-70 mm.

In an embodiment, the tube has an internal diameter in the range 5 mm-20 mm, preferably 10 mm-15 mm.

In an embodiment, the tube has a thickness in a range 100 μm-300 μm, preferably 175 μm-225 μm.

In an embodiment, both the tube and the muff are biocompatible and bioresorbable materials.

In an embodiment, wherein the implant is cylindrical.

In an embodiment, the muff is a material selected in the group of Polyglycolide-co-lactide (PGLA), Polydioxanone (PDO), Poly-L-lactic acid (PLLA) and/or Polycaprolactone (PCL).

In an embodiment, the muff has a thickness in a range 200 μm-1200 μm, preferably about 1000 μm.

In an embodiment, the tube is a polymeric substrate, preferably a polymer selected in the group of chitosan, poly (lactic-co-glycolic acid), polycaprolactone, and mixtures thereof.

In an embodiment, wherein the gel comprises chitosan, collagen or their mixtures; or synthetic extracellular matrix.

In an embodiment, the electrodes are made of metal, preferably gold.

In an embodiment, the electrodes are interdigitated comb electrodes each electrode comprises a bus bar that holds a row of pins along said bus bar.

In an embodiment, the electrodes have a length I in the range 10 mm-25 mm, preferably 14 mm-18 mm. In particular,
the distance between two pins in the same electrode is in the range 25 μm-200 μm, preferably 50 μm-150 μm, more preferably 75 μm-125 μm; and/or
each pin has a thickness in the range 10 μm-30 μm, preferably in the range 15 μm-25 μm; and/or
each pin has a length about the perimeter of the section of the internal surface of the tube, or a length in the range 2.5 mm-3.5 mm.

This invention also relates to a method for repairing a nerve comprising the following steps:
introducing the nerve to be repaired inside an implant as disclosed hereabove, through the gel such that the axon is placed inside the channel,
applying a voltage between the electrodes.

### Definitions

In the present invention, the following terms have the following meanings:

“Axon” refers to the long extension of a nerve cell, that typically conducts electrical impulses known as action potentials away from the nerve cell body. These impulses induce at the end of the extension called synapse the release of a substance, the neurotransmitter which delivers an information to the target cell. During cell growth, a neurite forms first, which becomes an axon after sufficient growth. In this disclosure, neurite and axon are used indistinctly.

“Injury”: refers to refers to a pathological damage caused by immediate physical stress. In the context of this disclosure, an injury results in the cut of a nerve, and in a gap between the two ends of the severed nerve, interrupting the nerve impulses.

### DETAILED DESCRIPTION

The following detailed description will be better understood when read in conjunction with the drawings. For the purpose of illustration, the device is shown in the preferred embodiments. It should be understood, however that the application is not limited to the precise arrangements, structures, features, embodiments, and aspect shown. The drawings are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

The present disclosure is a contactless electroactive device to be used as a neural implant in the case of an injury of the subject's nerve and where the subject's nerve was cut. The implant is intended for use in the repair of nerve damage, more precisely on the axon, enabled by its advantageous ability to guide and accelerate axon regrowth. Furthermore, the stimulation configuration avoids direct contact between the electrodes and the cells and/or the culture medium and prevents the creation of ions, local temperature increases and pH modification.

The present disclosure relates to a nerve repair implant (1) comprising a tube (2), at least two electrodes (3), a muff (4) and a gel (5).

FIG. 1 shows an implant (1) according to the disclosure.

As shown in FIG. 1, the implant (1) comprises an insulating tube (2) forming a channel, said channel comprising a gel (5), at least two electrodes (3) designed on the surface of the tube (2), and an insulating muff (4) surrounding the tube (2).

In an embodiment, the implant (1) is preferably adapted to repair nerve (6) from the peripheral nervous systems.

In an embodiment, the implant (1) may be made in a cylindrical shape, which matches the shape of an axon, as shown in FIG. 2, and allows a better stimulation growth and/or repair.

The gel (5) is adapted to accommodate an axon and ensure growth of the axon and its growth cone. The diameter of the gel should be about 5 mm to 20 mm. Indeed, the largest section of sciatic nerve may reach 20 mm in diameter. The diameter of the gel (5) is selected according to the nerve (6) to be rebuild. Preferably, the diameter of the gel may be about 10 mm to 15 mm.

In an embodiment, the gel (5) fills the channel formed by the tube (2).

In an embodiment, the gel (5) coats the internal surface of the tube (2).

In an embodiment, the gel (5) is preferably made of collagen, chitosan or their mixtures. Particularly suitable chitosan has an acetylation degree no higher than 20% and is used at a concentration in the hydrogel preferably from 0.25% to 5% relative to the total weight of the hydrogel, as disclosed in international patent application WO2014013188, the full content of which is included by reference. The gel (5) may be also a synthetic extracellular matrix, such as such as those described by Aisenbrey and Murphy, in Synthetic alternatives to Matrigel, Nat Rev Mater, 2020, the full content of which is included by reference.

Advantageously, the gel (5) provides a favorable growth environment compatible with the implementable living environment, preferably an in vivo cell growth environment.

The insulating tube (2) has an internal surface, an external surface and a thickness.

The tube (2) has preferably a length in the range 15 mm-85 mm, preferably 30 mm-70 mm. The length of the tube (2) is selected according to the size of the injury, i.e., to the distance between the two ends of the cut nerve or to the distance between the nerve extremity and the muscle to which the nerve should reconnect.

The tube (2) has preferably an internal diameter in the range 5 mm-20 mm, preferably 10 mm-15 mm. The internal diameter of the tube (2) is obviously the same as the diameter of the gel (5). The tube (2) has preferably a thickness in a range 100 μm-300 μm, preferably 175 μm-225 μm. The tube (2) thickness allows to insulate the electrodes (3) from the gel (5) and prevent the electrodes (3) to be in a direct electric contact with the gel (5).

Advantageously, the tube (2) internal diameter is adjusted to the diameter of the nerve (6) to repair, so that the internal diameter of the tube (2) is 10%-40% oversized compared to the nerve diameter.

In an embodiment, the tube (2) is preferably made from a polymeric substrate, preferably a polymer selected in the group of chitosan, Poly (lactic-co-glycolic acid), Polycaprolactone, and mixtures thereof.

Advantageously, the tube is made preferably from at least one dielectric material. The tube (2) may be made from biocompatible and bioresorbable materials, that will resorb in vivo. More preferably, the bioresorbable material is selected to have a resorption rate that matches that of axons growth: a typical resorption rate of 0.5 mm/day is suitable.

The electrodes (3) are designed on the external surface of the tube (2). Preferably, the electrodes (3) are designed to be deployed on the maximum external surface of the tube (2), so that the EF will spread over the whole tube surface.

One edge of each electrode (3) may be linked to the battery so as to power the device.

In an embodiment, one edge of each electrode (3) preferably terminates in a pole end (32) with a width in the range 1 mm-10 mm, preferably 5 mm, and a thickness in the range 2 mm-5 mm, preferably 3 mm. The pole end (32) may be linked to the battery so as to power the device.

In an embodiment, as illustrated in FIG. 3 and FIG. 4 the electrodes (3) are preferably interdigitated comb electrodes with a length L in the range 15 mm-30 mm, preferably 20 mm-25 mm, and each electrode (3) comprising a bus bar (30) with a length I in the range 10 mm-25 mm, preferably 14 mm-18 mm, said bus bar (30) holding a row of pins (31). Each bus bar (30) is terminated with a pin (31) on one side and a pole end (32) on the other side for easy connection to the voltage generator The electrodes are interdigitated so that the pins (31) of each electrode (3) are alternated.

In an embodiment, pins (31) are alternated in a periodic and regular manner, with a constant distance d between two successive pins (31) on an electrode (3), as shown on FIG. 4. The distance d between two successive pins (31) on a same bus bar (30) of the same electrode (3) is preferably in the range 25 μm-200 μm, preferably 50 μm-150 μm, more preferably 75 μm-125 μm.

In an embodiment, each electrode (3) consists of a first zones with a distance between two successive pins (31) on a same bus bar (30) d1 and a second zone with a distance between two successive pins (31) on a same bus bar (30) d2, d1 and d2 being in the range 25 μm-200 μm, preferably 50 μm-150 μm, more preferably 75 μm-125 μm.

Alternatively, the distance d between two successive pins (31) on a same bus bar (30) is not constant. Distance d may be increasing regularly-linearly or logarithmically or exponentially for instance-along the bus bar (30). In this configuration, distance d is preferably varying in the range 25 μm-500 μm.

Preferably, the distance between the extremity of the pins (31) of an electrode (3) and the bus bar (30) of the other electrode (3) is larger than or equal to the shortest distance d, d1, d2 as defined hereabove This distance avoids creating an electric field of high intensity associated with sharp tips.

Preferably, the tube (2) is longer than L, so as to facilitate suturing of the tube (4) to the subject's tissues on both sides

In an embodiment, the pins (31) are substantially parallel to each other, and each pin (31) is substantially perpendicular to the bus bar (30).

In an embodiment, the pins (31) are not parallel to each other, periodically or otherwise, and some pins (31) are not perpendicular to the bus bar (30). This allows for a locally variable electrical field application.

When the tube (2) is cylindric and supporting two interdigitated electrodes (3), the angle α between the two bus bars (30) is preferably larger than 180°, more preferably larger than 275°, so that the pins (31) cover a large surface of the tube (2). Here, angle α corresponds to the sector of the cylindric tube (2) over which the pins (31) are laid, as shown on FIG. 3.

In an embodiment, each pin (31) has a thickness in the range 10 μm-30 μm, preferably in the range 15 μm-25 μm, and each pin (31) has a length about the perimeter of the section of the internal surface of the tube (2), or a length in the range 2.5 mm 3.5 mm.

In an embodiment, the electrodes (3) are preferably made of metal, preferably gold, a biocompatible and a bioresorbable material, that will resorb in vivo.

Electrodes (3) may be prepared on the external surface of the tube (2) by photolithography. In a first method, the electrodes are prepared on a planar insulating substrate, then the substrate is twisted to form the tube (2) with electrodes on its external surface. In a second method, the electrodes are directly prepared on the tube (2) made of insulating material.

Advantageously, the interdigitated geometry of the comb electrodes (3) produces a null current, as in the global electrical field.

The electrodes (3) and the gel (5) are separated by the solid surface of the tube (2).

An insulating muff (4) is surrounding the tube (2) and in direct contact with the tube (2) and the electrodes (3).

In an embodiment, the muff (4) has preferably a thickness in a range 200 μm-1200 μm, preferably about 1000 μm.

In an embodiment, the muff (4) is preferably made from a material selected in the group of Polyglycolide-co-lactide (PGLA), Polydioxanone (PDO), Poly-L-lactic acid (PLLA) and/or Polycaprolactone (PCL), preferably a Polycaprolactone. The material is preferably an insulating, biocompatible and non-toxic material, with a controlled bioresorbable kinetic so that the muff (4) resorbs at a rate that matches that of axon growth.

In one embodiment, the muff (4) is preferably 10% oversized compared to the tube (2), the electrodes (3) and the gel (5) construction and extends on both ends to be used for tissue connectivity.

Advantageously, the muff (4) allows to cover and insulate the electrode (3) from the in vivo environment and ensure the tissue connectivity.

The following configuration is especially suitable for electrodes (3): two comb electrodes (3) are used in the implant (1). The electrodes (3) are built preferably on a tubular substrate by photolithography so that the electrodes (3) are printed in a semi-tubular shape or arc shape, as illustrated in FIG. 3, so that the EF spread over the whole tube. The electrodes (3) are composed in each opposite edge of each electrode (3) by a pole end (32). From the pole end (32) merges a bus bar (30) with a length in a range 10 mm-25 mm, preferably 18 mm-14 mm, preferably 16 mm, and a separation angle “α” between two bus bars (30) in a range 180°-355°, preferably 275°-350°. The bus bar (30) holds a row of pins (31) along and perpendicular to the bus bar (30), with pins (31) distributed regularly along the bus bar (30).

As aforementioned, the implant (1) is configured to be implanted around an injured axon of a subject's nerve, said axon being cut, to insure the axon repair and regrowth.

The implant (1) may be implanted via a nerve sparing reimplantation procedure. The implant (1) may be any variant as disclosed hereabove.

The method for repairing a nerve (6) by implanting the implant (1) during a nerve sparing procedure comprises the following steps:
first, the nerve (6) to be repaired is introduced inside an implant (1), through the gel (5) such that the axon is placed inside the channel.
second, each electrode (3) is connected to a battery, so as to be able to generate a positively charged electrode (3) and a negatively charged electrode.
then, a voltage is applied between the electrodes (3).

The voltage may be applied during a time length in a range 2 h-72 h, preferably, 4 h-48 h, more preferably 8 h.

The applied voltage may be constant, for instance in the range 4 V-20 V, preferably 9 V-18 V, more preferably 9 V. Alternatively, the applied voltage may be an alternative voltage, with a frequency in the range 10 Hz-500 Hz, preferably 100 Hz-250 Hz, more preferably 150 Hz-200 Hz. The amplitude of alternative voltage may be in the range 4 V-20 V, preferably 9 V-18 V, more preferably 9 V.

The applied voltage may be varying in time, in amplitude and/or in frequency. In some embodiments, the applied voltage is a sweep ramp of voltage, either increasing or decreasing, in the ranges of amplitude listed hereabove. In some embodiments, the frequency of applied voltage is a sweep range in the ranges of frequencies listed hereabove.

The following set-up for voltage has been found especially suitable to promote growth of an axon: 9 V during 8 h. With this set-up, a growth of 150 μm or more is usually observed on an axon.

Without being bound by theory, it has been observed that application of Electrical Fields (EF) on the tube (2) induces a spatial distribution of the energy of adhesion, which is critical for neuritogenesis. More precisely, it seems that the relative importance of the Lifshitz-Van der Walls contribution (dispersive, long-range interactions) and non-dispersive contribution (short-range interactions) governs neuritogenesis. For instance, it has been observed that a stimulation of 24 hours with a voltage of 9 V with electrodes set-up of example 1 leads to an increase of the dispersive part of surface energy (from 3.3 mN. m ⁻¹ to 10.9 mN. m⁻¹), which becomes greater than the non-dispersive part of the surface energy (from 18.7 mN. m⁻¹ to 10.0 mN. m⁻¹). The materials selected for the tube (2) preferably present a dispersive part of surface energy higher than 40% of the whole surface energy, preferably higher than 50% of the whole surface energy.

After stimulation of the axon during 8, a growth of 92 μm is observed on the axon which represents 108 % of the axon initial length.

In an embodiment, a pre-stimulation is applied on the substrate (2) before the implantation. The features of pre-stimulation voltage are the same as the simulation voltage disclosed hereabove. Once the pre-stimulated implant (1) is implanted, the voltage is applied between the electrodes (3) as described hereabove.

In an embodiment, the voltage preferably applied between the electrodes (3) for a pre-stimulation is 18 V during 48 h.

It is noteworthy that pre-stimulation is applied to the implant (1) before placing the gel (5) in the implant (1). This set-up seems to predispose the surfaces of the implant (1) leading to a specific arrangement of the constituent of the gel (5) in the implant (1) characterized by local gradients of adhesion energy. These gradients remain at the surface, even after removal of the electrical fields: the surface modification is thus permanent and orient continuously neurite/axon growth. Stimulation made immediately after placing the gel (5) in the implant (1) seems to lead to the same permanent effect on the surface and therefore on the orientation of neurite/axon growth.

The present disclosure also relates to a set of at least two electrodes (3) designed to be laid in direct contact on the external surface of a tube (2), said electrodes being interdigitated comb electrodes with a length L in the range 15 mm-30 mm, preferably 20 mm-25 mm, and each electrode (3) comprising a bus bar (30) with a length I in the range 10 mm-25 mm, preferably 14 mm-18 mm, said bus bar (30) holding a row of pins (31). The electrodes are interdigitated so that the pins (31) of each electrode (3) are alternated.

In an embodiment, pins (31) are alternated in a periodic and regular manner, with a constant distance d between two successive pins (31) on an electrode (3), as shown on FIG. 4. The distance d between two successive pins (31) on a same bus bar (30) of the same electrode (3) is preferably in the range 25 μm-200 μm, preferably 50 μm-150 μm, more preferably 75 μm-125 μm.

Preferably, the distance between the extremity of the pins (31) of an electrode (3) and the bus bar (30) of the other electrode (3) is larger than or equal to the distance d. This distance avoids creation of an electric field of high intensity associated with sharp tips.

The other features disclosed hereabove for the electrodes (3) designed on the external surface of a tube (2) are also suitable for the set of electrodes (3) designed to be laid in direct contact on the external surface of a tube (2).

### BRIEF DESCRIPTION OF THE DRAWINGS

FIG. 1 is a schematic of a nerve repair implant (1) according to an embodiment showing an insulating tube (2), two electrodes (3), an insulating muff (4) and a gel (5).

FIG. 2 is a schematic showing a nerve (6) placed inside an implant (1) according to an embodiment.

FIG. 3 is a schematic of electrodes (3) according to an embodiment designed on the external surface of the tube (2) showing two interdigitated comb electrodes, where each electrode (3) is consisting of a bus bar (30) that holds a row of pins (31).

FIG. 4 is a planar schematic of electrodes (3) according to an embodiment showing two interdigitated comb electrodes, where each electrode (3) is consisting of a bus bar (30) that holds a row of pins (31) and terminates with a pole end (32).

FIG. 5 is a schematic showing a nerve (6) and nervous system inside a human body.

FIG. 6 is a photograph showing nervous cell culture in a reference environment, without electrical field applied. On the left, culture is imaged by phase contrast microscopy after 1 day of growth. On the right, the image is treated to show only cells.

FIG. 7 is a photograph showing nervous cell culture in a reference environment, with electrical field applied. Two electrodes are visible (vertical grey bars on left and right). The electrical field has been applied with a pre-stimulation of 72 h (9 V) of the substrate before deposition of the cells, then a stimulation of 9 V during 4 h. Culture is imaged by phase contrast microscopy after one day of growth.

FIG. 8 is the treatment of FIG. 7 to show only cells and growing neurites, pointed by dotted arrows. Neurites preferably grow in the direction of electrical field, i.e., perpendicular to electrodes here.

FIG. 9 is a photograph showing nervous cell culture in a reference environment, with electrical field applied. one electrode is visible (oblique grey bar). The electrical field has been applied with a pre-stimulation of 72 h (9 V) of the substrate before deposition of the cells, then a stimulation of 9 V during 4 h (same as for FIG. 7). Culture is imaged by phase contrast microscopy after eight days of growth.

FIG. 10 is the treatment of FIG. 9 to show only cells and growing neurites, pointed by dotted arrows. Neurites preferably grow in the direction of electrical field, i.e., perpendicular to electrodes here.

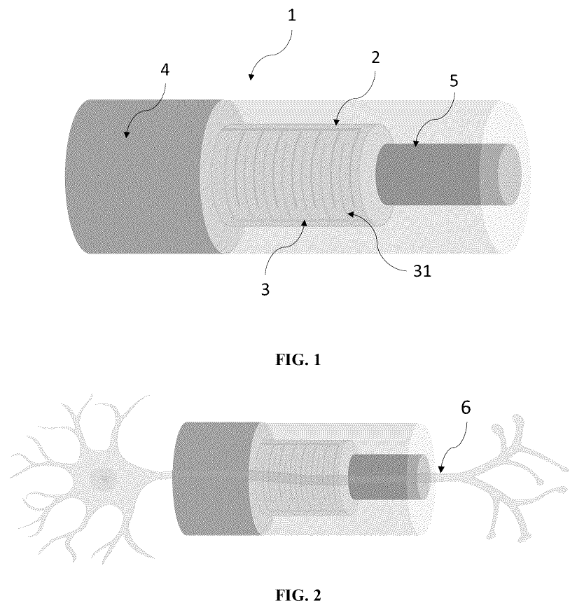

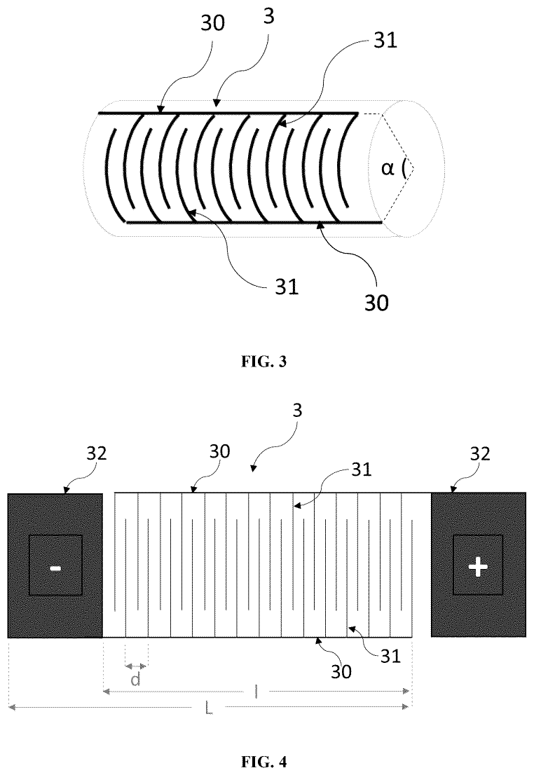

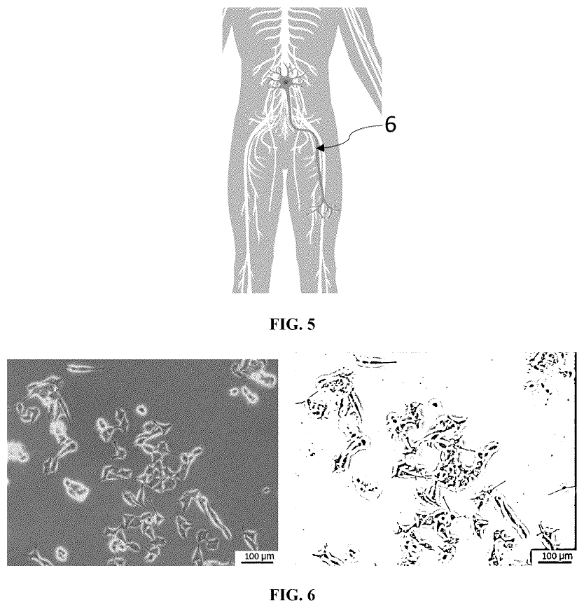

### EXAMPLES

The present invention is further illustrated by the following examples.

### Example 1:

An implant (1) made in a cylindrical shape comprises an insulating tube (2) made from chitosan with a length of 30 mm, having an internal surface and an external surface, said tube (2) forming a channel with an internal diameter of 4 mm, said channel comprising a gel (5) made from collagen which fills the channel. Two gold interdigitated comb electrodes (3) are designed on the external surface of the tube (2), with a length of 15 mm, and each electrode (3) consisting of a bus bar (30) that holds a row of pins (31) separated by a distance of 200 μm where each pin is 20 μm thick, and 9.5 mm long. One edge of each electrode (3) terminates in a pole end of 5 mm wide, and 3 mm thick connected to a battery. An insulating muff (4) made from polycaprolactone with 33 mm in length and 600 μm thick surrounds the tube (2) in direct contact with the tube (2). The electrodes (3) and the gel (5) are separated by 200 μm of the insulating tube (2).

Comb electrodes (3) are printed by photolithography according to the following protocol. Insulating material is cleaned during 40 s in an acetone bath with ultrasound. Then insulating material is rinsed with isopropanol and dried under nitrogen. A resin is then spin-coated on the insulating material and cured at 100° C. A mask according to the expected structure of electrodes is laid on the resin, and UV-light is applied. Then, a developer (AZ351B from Clariant) is used: the coated insulated material is immersed in developer then in water and dried. Finally, gold is deposited by sputtering with Argon plasma. To remove excess deposit of gold, the substate is treated in an ultrasound bath. Finally, the insulating material is twisted to form the tube (2) with expected dimensions.

A pre-stimulation is applied to the implant (1) for 72h, with 9 V.

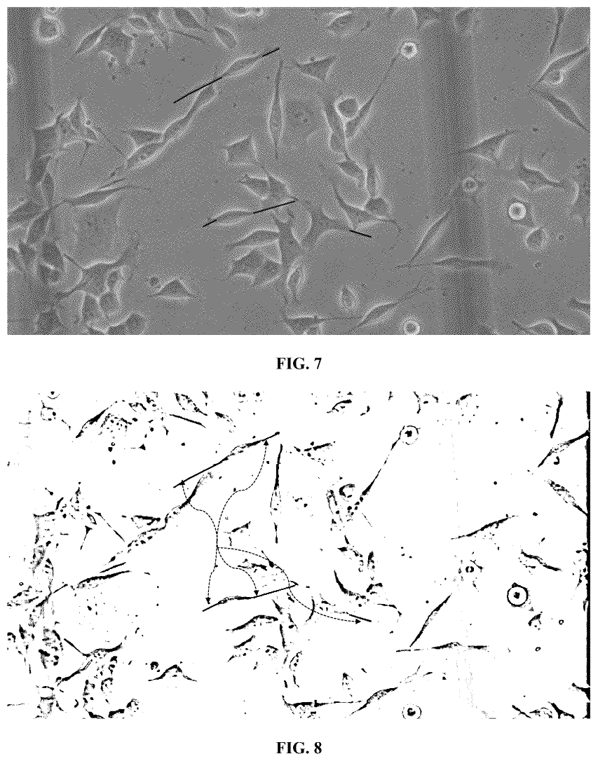

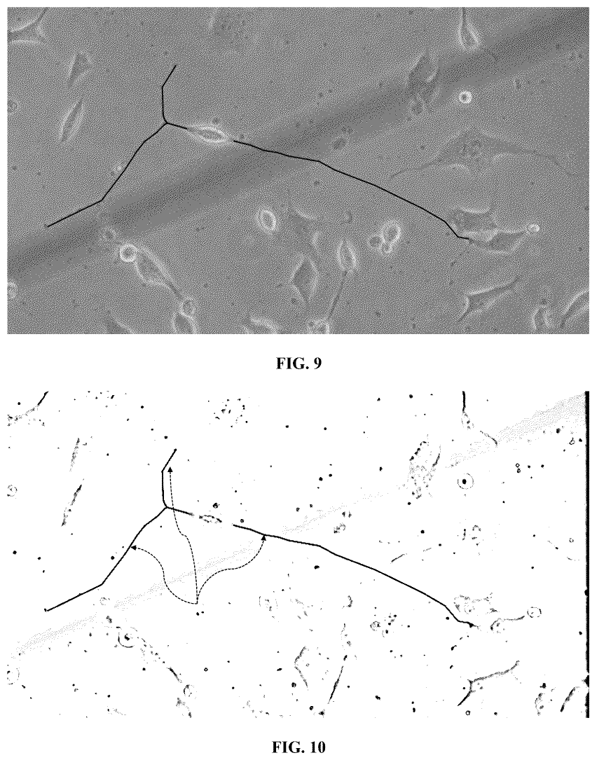

Then a nerve (6) (NSC-34 cells from mouse motoneurons) is first introduced in the implant (1), through the gel (5), second, the pole of each electrode (3) is connected to a battery, third, anchoring stitches are made at both ends of the tube to maintain the device in the tissue. Then, 9 V is applied between the electrodes (3), during 4 h to stimulate the cells and the cells are let for growth, evidenced by growth of axons (or neurites). After 24 h, neurites have grown in the direction of electrical field applied. A growth of about 20 μm is observed, as illustrated on FIGS. 7 and 8. After 8 days, a growth of 100 μm is measured, as illustrated on FIGS. 9 and 10. Growth up to 150 μm are commonly observed.

### Comparative Example

NSC-34 axons from mouse motoneurons are cultured in the same environment as in example 1, except that electrical field (pre-stimulation and stimulation) is not applied. FIG. 6 shows the cells after 1 day of growth. Neurites are not oriented, and growth is limited, less than 20 μm.

### Example 2

Experience of example 1 is reproduced, in which the distance between two pins of the same electrode is 50 μm. Similar growth results are obtained.

### Example 3

Experience of example 1 is reproduced, in which the gel (5) is coating the internal surface of the tube (2). Similar growth results are obtained.

### Example 4

Experience of example 1 is reproduced, in which the insulating tube (2) is made from a poly (lactic-co-glycolic acid). Similar growth results are obtained.

Example 5

Experience of example 1 is reproduced, in which the insulating tube (2) is made from a polycaprolactone. Similar growth results are obtained.

Example 6

Experience of example 1 is reproduced, in which the insulating tube (2) is made from chitosan and poly (lactic-co-glycolic acid). Similar growth results are obtained.

### Example 7

Experience of example 1 is reproduced, in which the insulating tube (2) is made from chitosan and polycaprolactone. Similar growth results are obtained.

### Example 8

Experience of example 1 is reproduced, in which the insulating tube (2) is made from poly (lactic-co-glycolic acid) and polycaprolactone. Similar growth results are obtained.

### Example 9

Experience of example 1 is reproduced, in which the gel is made from a collagen. Similar growth results are obtained.

### Example 10

Experience of example 1 is reproduced, in which the gel is made from a synthetic extracellular matrix. Similar growth results are obtained.

### Example 11

Experience of example 1 is reproduced, in which the gel is made from chitosan and collagen. Similar growth results are obtained.

### Example 12

Experience of example 1 is reproduced, in which the gel is made from chitosan and a synthetic extracellular matrix. Similar growth results are obtained.

Example 13

Experience of example 1 is reproduced, in which the insulating muff (4) is made from polyglycolide-co-lactide. Similar growth results are obtained.

### Example 14

Experience of example 1 is reproduced, in which the insulating muff (4) is made from polydioxanone. Similar growth results are obtained.

### Example 15

Experience of example 1 is reproduced, in which the insulating muff (4) is made from poly-L-lactic. Similar growth results are obtained.

### Example 16

Experience of example 1 is reproduced, in which the insulating muff (4) is made from polycaprolactone. Similar growth results are obtained.

### REFERENCES

1 implant | 2 insulating tube | 3 electrodes | 4 insulating muff | 5 gel | 6 nerve | 30 bus bar | 31 pin | 32 pole end.

1. A nerve repair implant comprising:
an insulating tube having an internal surface and an external surface, said tube forming a channel, said channel comprising a gel,
at least two electrodes designed on the external surface of the tube,
an insulating muff surrounding the tube and in direct contact with the tube,
wherein the gel is adapted to accommodate an axon and ensure growth of the axon, and
wherein the electrodes and the gel are separated by the tube.

2. The implant according to claim 1, wherein the gel fills the channel or the gel coats the internal surface of the tube.

3. The implant according to claim 1, wherein the tube has a length in the range 15 mm-85 mm,

4. The implant according to claim 1, wherein the tube has an internal diameter in the range 5 mm-20 mm.

5. The implant according to claim 1, wherein the tube has a thickness in a range 100 μm-300 μm.

6. The implant according to claim 1, wherein both the tube and the muff are biocompatible and bioresorbable materials.

7. The implant according to claim 1, wherein the implant is cylindrical.

8. The implant according to claim 1, wherein the muff is a material selected in the group of Polyglycolide-co-lactide, Polydioxanone, Poly-L-lactic acid and/or Polycaprolactone.

9. The implant according to claim 1, wherein the muff has a thickness in a range 200 μm-1200 μm,

10. The implant according to claim 1, wherein the tube is a polymeric substrate.

11. The implant according to claim 1, wherein the gel comprises chitosan, collagen or their mixtures; or synthetic extracellular matrix.

12. The implant according to claim 1, wherein the electrodes are made of metal,

13. The implant according to claim 1, wherein the electrodes are interdigitated comb electrodes each electrode comprises a bus bar that holds a row of pins along said bus bar.

14. The implant according to claim 1, wherein the electrodes have a length l in the range 10 mm-25 mm,

15. The implant according to claim 14, wherein the distance between two pins in the same electrode is in the range 25 μm-200 μm,

16. The implant according to claim 14, wherein each pin has a thickness in the range 10 μm-30 μm,

17. The implant according to claim 14, wherein each pin has a length about the perimeter of the section of the internal surface of the tube, or a length in the range 2.5 mm-3.5 mm.

18. Method for repairing a nerve comprising the following steps:
introducing the nerve to be repaired inside an implant according to claim 1, through the gel such that the axon is placed inside the channel,
applying a voltage between the electrodes.

19. The implant according to claim 10, wherein the tube is a polymeric substrate, selected in the group of chitosan, poly (lactic-co-glycolic acid), polycaprolactone, and mixtures thereof.

20. The implant according to claim 12, wherein the electrodes are made of gold.
